(19) United States
(12) Patent Application Publication
(10) Pub. No.: US 2022/0139253 A1
(43) Pub. Date: May 5, 2022

(54) GUIDANCE SYSTEM AND METHOD FOR ACTION POSTURES

(71) Applicant: INSTITUTE FOR INFORMATION INDUSTRY, Taipei City (TW)

(72) Inventors: Heng-Yi CHEN, Taipei (TW); Rong-Sheng WANG, Taipei (TW)

(73) Assignee: INSTITUTE FOR INFORMATION INDUSTRY, Taipei City (TW)

(21) Appl. No.: 17/109,363

(22) Filed: Dec. 2, 2020

(30) Foreign Application Priority Data
Nov. 3, 2020 (TW) 109138241

(51) Int. Cl. G09B 19/00; G06K 9/00; G06K 9/62; G11B 27/031

(52) U.S. Cl. CPC G09B 19/0038; G11B 27/031; G06K 9/6202; G06K 9/00342

(57) ABSTRACT

A guidance system and method for action postures are provided. The guidance system includes a first skeleton recognition model, an editing device and a storage device. The first skeleton recognition model is used for generating a standard skeleton video according to a standard action posture video. The standard skeleton video includes a standard specific action posture of a person, and the standard skeleton video includes several skeleton pictures for standard specific action posture corresponding to the standard specific action posture. The editing device is used for receiving a command of an editor to generate at least one action posture guidance information corresponding to the skeleton pictures for standard specific action posture. The storage device is used for storing the skeleton pictures for standard specific action posture and the at least one action posture guidance information corresponding to the skeleton pictures for standard specific action posture.

This application claims the benefit of Taiwan application Serial No. 109138241, filed Nov. 3, 2020, the subject matter of which is incorporated herein by reference.

### BACKGROUND OF THE INVENTION

### Field of the Invention

The invention relates in general to a guidance system and method, and more particularly to a guidance system and method for action postures.

### Description of the Related Art

In recent years, the demand for fitness coaches gradually increases due to the increase in sports population. Furthermore, along with the development in technology, many exercise-related digital audio/video contents and applications are provided so that people can do exercise and training at home.

When it comes to exercise and training, the most important issue is whether the action postures are correct. However, the exercise-related digital audio/video contents and applications currently available in the market only provide learning videos for the users to view but cannot provide any feedback with respect to the users' action postures. Therefore, the users can only view the videos for self-learning and cannot determine whether their action postures are correct or not and thus have no idea about how to make improvement.

Some applications can recognize the users' action postures but require building and training a model based on many action postures beforehand, and the cost and efforts for building such an action recognition model is huge. Most fitness coaches or persons who play exercise-related digital audio/video contents on the Internet cannot afford it. Besides, digital audio/video contents of different exercise may require different action recognition models. However, the fitness coaches or persons who play the digital audio/video contents on the Internet seek to diversify the exercise contents to attract people, and thus generate a large amount of digital audio/video contents. It is extremely difficult to build individual action recognition model for each digital audio/video content.

Therefore, it has become a prominent task for the industry to provide a convenient system and tool allowing fitness coaches or persons who play exercise-related digital audio/video contents on the Internet to edit the action posture guidance information with respect to the users' actions, which often require guidance, according to the standard exercise video. When the user is doing exercise and training according to the exercise video, the system and tool can provide corresponding action posture guidance information to the user whose action posture is determined as incorrect for helping the user to achieve a desired effect of exercise and training and to avoid having sport injury due to incorrect action postures.

### SUMMARY OF THE INVENTION

The invention is directed to a guidance system and method for action postures capable of determining whether the user's action posture is correct according to the position, the angle and the speed of the action posture when the user is doing exercise or training, and providing the user with action posture guidance information which helps the user to avoid having incorrect action postures during the exercise or training process or ending up with an undesired effect or sports injury.

According to one embodiment of the present invention, a guidance system for action postures is provided. The guidance system includes a first skeleton recognition model, an editing device and a storage device. The first skeleton recognition model is used for generating a standard skeleton video according to a standard action posture video. The standard skeleton video includes a standard specific action posture of a person, and the standard skeleton video includes several skeleton images for standard specific action posture corresponding to the standard specific action posture. The editing device is used for receiving a command of an editor to generate at least one action posture guidance information corresponding to the skeleton images for standard specific action posture. The storage device is used for storing the skeleton images for standard specific action posture and the at least one action posture guidance information corresponding to the skeleton images for standard specific action posture.

According to another embodiment of the present invention, a guidance method for action postures is provided. The guidance method includes the following steps. A standard skeleton video is generated according to a standard action posture video, wherein the standard skeleton video includes a standard specific action posture of a person, and the standard skeleton video includes several skeleton images for standard specific action posture corresponding to the standard specific action posture. A command of an editor is received to generate at least one action posture guidance information corresponding to the skeleton images for standard specific action posture. The skeleton images for standard specific action posture and the at least one action posture guidance information corresponding to the skeleton images for standard specific action posture are stored.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 is a block diagram of a guidance system for action postures according to an embodiment of the present invention;

FIG. 2 is a flowchart for establishing a guidance knowledge base according to an embodiment of the present invention;

FIG. 3 is a schematic diagram of a standard action posture video according to an embodiment of the present invention;

FIG. 4 is a schematic diagram of a standard skeleton video according to an embodiment of the present invention;

FIG. 5 is a schematic diagram of an editing device according to an embodiment of the present invention;

FIG. 6 is a flowchart of a guidance method for action postures according to an embodiment of the present invention;

FIG. 7 is a schematic diagram of a user live streaming according to an embodiment of the present invention;

FIG. 8 is a schematic diagram of a user action posture skeleton video according to an embodiment of the present invention;

FIG. 9 is a schematic diagram showing that the skeleton picture for user specific action posture is compared with the skeleton image for standard specific action posture to generate a comparison result according to an embodiment of the present invention; and

FIG. 10 is a schematic diagram of a user action posture skeleton video according to another embodiment of the present invention.

### DETAILED DESCRIPTION OF THE INVENTION

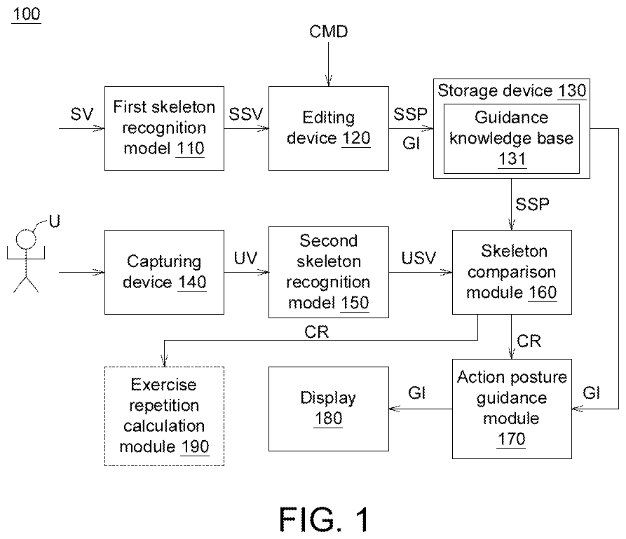

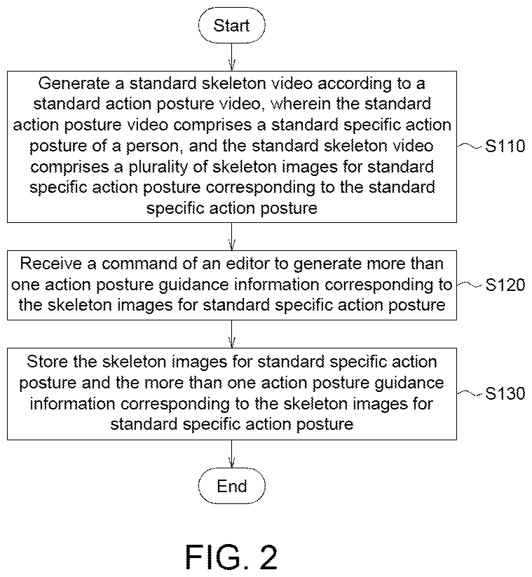

Referring to FIGS. 1 and 2, FIG. 1 is a block diagram of a guidance system for action postures 100 according to an embodiment of the present invention, and FIG. 2 is a flowchart for establishing a guidance knowledge base according to an embodiment of the present invention. The guidance system for action postures 100 includes a first skeleton recognition model 110, an editing device 120 and a storage device 130. The first skeleton recognition model 110 can be a deep learning model. The editing device 120 can be an editing software. The storage device 130 can be a conventional hard disc (HDD) or a solid-state disc (SSD). The storage device 130 includes a guidance knowledge base 131.

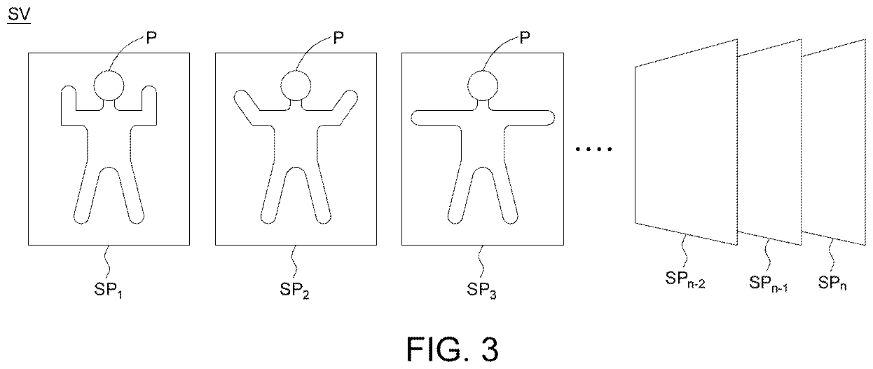

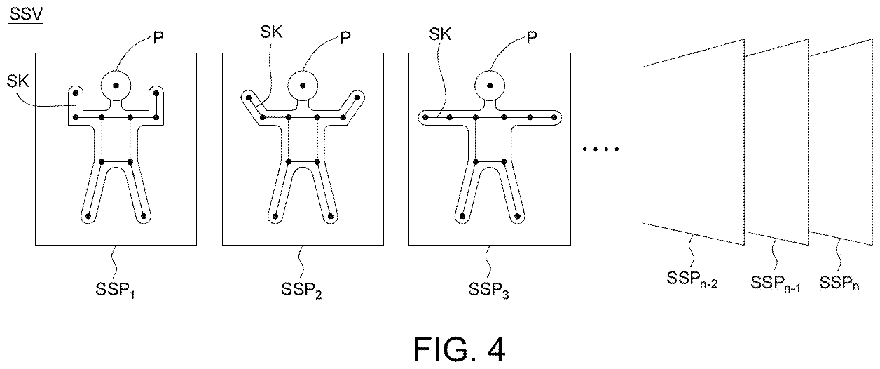

In step S110, a standard skeleton video SSV is generated by the first skeleton recognition model 110 according to a standard action posture video SV. The standard action posture video SV includes a standard specific action posture of a person, and the standard skeleton video SSV includes several skeleton images for standard specific action posture SSP corresponding to the standard specific action posture. Referring to FIG. 3, a schematic diagram of a standard action posture video SV according to an embodiment of the present invention is shown. The standard action posture video SV can be a video of a standard specific action posture of a person P doing exercise or training. The standard action posture video SV includes several picture SP₁, SP₂, SP₃ . . . SPₙ₋₂, SPₙ₋₁ and SPₙ. Referring to FIG. 4, a schematic diagram of a standard skeleton video SSV according to an embodiment of the present invention is shown. The standard skeleton video SSV is a video with marked skeleton SK. The standard skeleton video SSV includes several skeleton images for standard specific action posture SSP₁, SSP₂, SSP₃ . . . SSPₙ₋₂, SSPₙ₋₁ and SSPₙ. The skeleton images for standard specific action posture SSP₁, SSP₂, SSP₃ . . . SSPₙ₋₂, SSPₙ₋₁ and SSPₙ correspond to the standard specific action posture. In an embodiment, the guidance system for action postures 100 further includes a communication module (not shown) for receiving the standard action posture video SV. The communication module can be a wired or wireless communication interface.

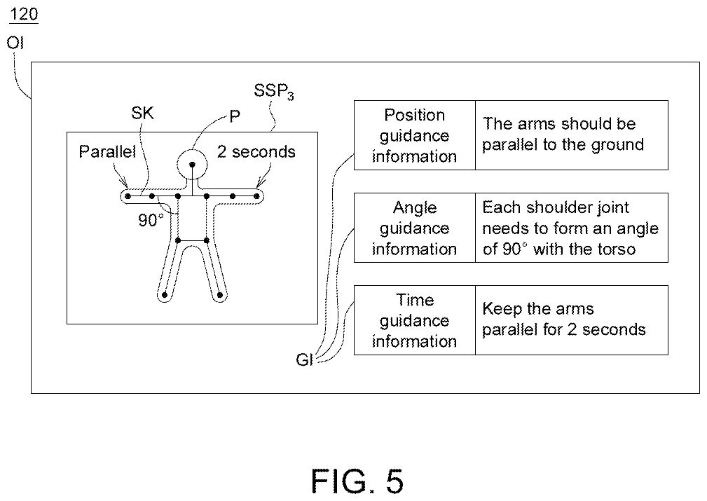

In step S120, a command CMD of an editor is received by the editing device 120 to generate at least one action posture guidance information GI corresponding to the skeleton image for standard specific action posture SSP. Each action posture guidance information GI includes at least one of a position guidance information, an angle guidance information, a time guidance information and a speed guidance information, or any combination of the above guidance information. Referring to FIG. 5, a schematic diagram of an editing device 120 according to an embodiment of the present invention is shown. The editing device 120 provides an operation interface OI on which a standard skeleton video SSV is displayed, the editor selects a skeleton image for standard specific action posture SSP from the standard skeleton video SSV and further marks the information on the selected skeleton image for standard specific action posture SSP. A skeleton SK in the selected skeleton image for standard specific action posture SSP includes several limbs and several joints. The information mark can be a position mark or a time mark on any of the limbs, or an angle mark on any of the joints and the limb connected thereto. For example, the operation interface OI of the editing device 120 displays the standard skeleton video SSV as shown in FIG. 4; the editor selects a skeleton image for standard specific action posture SSP₃ from the standard skeleton video SSV and performs information marking on the selected skeleton image for standard specific action posture SSP₃. Since the key point of the action in the skeleton image for standard specific action posture SSP₃ is the arm position, the duration of the arm action and the angle between the shoulder joint and the torso, the information mark done by the editor may include such as the position mark and time mark of the arm and the angle mark between and the shoulder joint and the torso. Then, the operation interface OI is further provided for the editor to edit the action posture guidance information GI according to the information mark. For example, the editor, according to the information mark, edits the position guidance information of the action posture guidance information GI as “the arms should be parallel to the ground”, edits the angle guidance information of the action posture guidance information GI as “each shoulder joint needs to form an angle of 90° with the torso” and edits the time guidance information of the action posture guidance information GI as “keep the arms parallel for 2 seconds”. In some other embodiments, the action posture guidance information GI may include a speed guidance information, such as “change the arms to a parallel position from a vertical position at a speed of 0.5 m/s”.

In step S130, the skeleton images for standard specific action posture SSP and the action posture guidance information GI corresponding to the skeleton images for standard specific action posture are stored to the storage device 130. The storage device 130 includes a guidance knowledge base 131 storing the skeleton images for standard specific action posture SSP and the action posture guidance information GI corresponding to the skeleton images for standard specific action posture. For example, the guidance knowledge base 131 stores the skeleton image for standard specific action posture SSP₃ and the action posture guidance information GI corresponding to the skeleton image for standard specific action posture SSP₃. In an embodiment, the guidance knowledge base 131 may store several skeleton images for standard specific action posture (such as SSP₁, SSP₂ and SSP₃) and the action posture guidance information GI corresponding to the skeleton images for standard specific action posture.

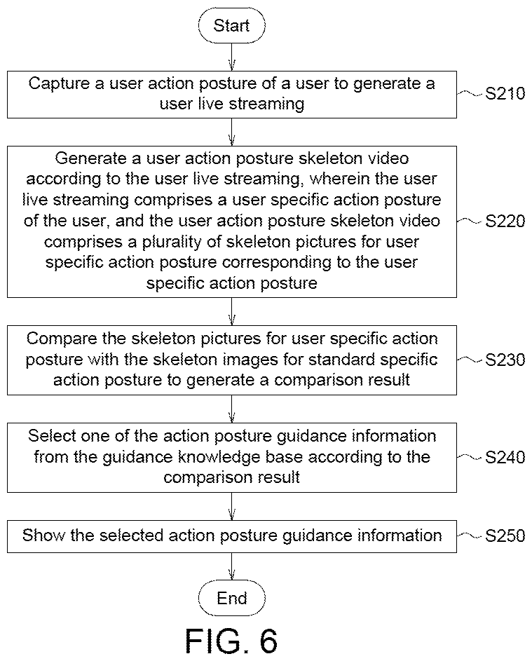

Referring to FIGS. 1 and 6, FIG. 6 is a flowchart of a guidance method for action postures according to an embodiment of the present invention. The guidance system for action postures 100 further includes a capturing device 140, a second skeleton recognition model 150, a skeleton comparison module 160, an action guidance module 170, and a display 180. The capturing device 140 can be a webcam of a mobile device, a computer's built-in lens or a fitness mirror lens. The second skeleton recognition model 150 can be similar or identical to the first skeleton recognition model 110. The second skeleton recognition model 150 can be a deep learning model. The skeleton comparison module 160 and the action guidance module 170 can be a processing circuit or a processing chip. The display 180 can be a screen of a mobile device, a liquid crystal screen of a computer, or a fitness mirror display.

In step S210, a user action posture of a user U is captured by a capturing device 140 to generate a user live streaming UV.

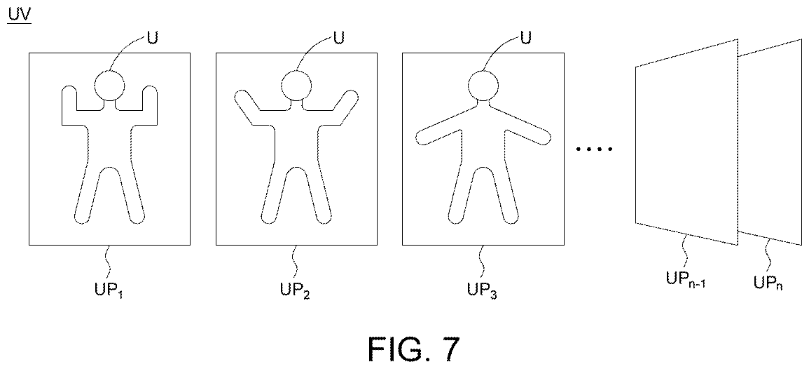

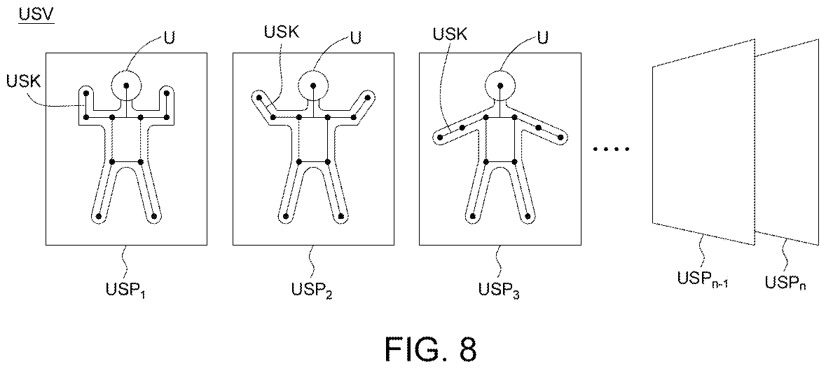

In step S220, a user action posture skeleton video USV is generated by the second skeleton recognition model 150 according to the user live streaming UV. The user live streaming UV includes a user specific action posture of the user U; the user action posture skeleton video USV includes several skeleton pictures for user specific action posture USP corresponding to the user specific action posture. Referring to FIG. 7, a schematic diagram of a user live streaming UV according to an embodiment of the present invention is shown. The user live streaming UV can be a video of a user specific action posture of the user U doing exercise or training according to the standard action posture video. The user live streaming UV includes several picture UP₁, UP₂, UP₃ . . . UPₙ₋₁ and UPₙ. Referring to FIG. 8, a schematic diagram of a user action posture skeleton video USV according to an embodiment of the present invention is shown. The user action posture skeleton video USV is a video with marked skeleton USK. The user action posture skeleton video USV includes several skeleton pictures for user specific action posture USP₁, USP₂, USP₃ . . . USPₙ₋₁ and USPₙ. The skeleton pictures for user specific action posture USP₁, USP₂, USP₃ . . . USPₙ₋₁ and USPₙ correspond to standard specific actions.

In step S230, the skeleton picture for user specific action posture USP is compared with the skeleton image for standard specific action posture SSP by the skeleton comparison module 160 to generate a comparison result CR. The comparison result CR includes a position comparison result, an angle comparison result, a time comparison result or a speed comparison result. The comparison result CR can also be any combination of the above comparison results. Furthermore, the skeleton comparison module 160 compares the position and angle between the skeleton USK in the user specific action posture skeleton picture USP and the skeleton SK in the skeleton image for standard specific action posture SSP to obtain a position comparison result and an angle comparison result, respectively. The skeleton comparison module 160 can obtain the start and the ending skeleton pictures of the user specific action posture of the user from several skeleton pictures for user specific action posture. For example, the user specific action posture is a user holding the dumb bells vertically and then extending his/her arms to be parallel to the ground. The user specific action posture skeleton picture USP₁ is the starting skeleton picture; the user specific action posture skeleton picture USP, is the ending skeleton picture. The duration for which the user performs the specific action can be calculated according to the user live streaming UV. The distance by which the user extends his/her hands can be calculated according to the positions of the user's palms at the skeletons in the skeleton pictures for user specific action posture USP₁ and USPₙ, respectively. The user's speed can be calculated according to the above time and distance. The user's time and speed can be compared with the time guidance information and the speed guidance information to obtain a time comparison result and a speed comparison result.

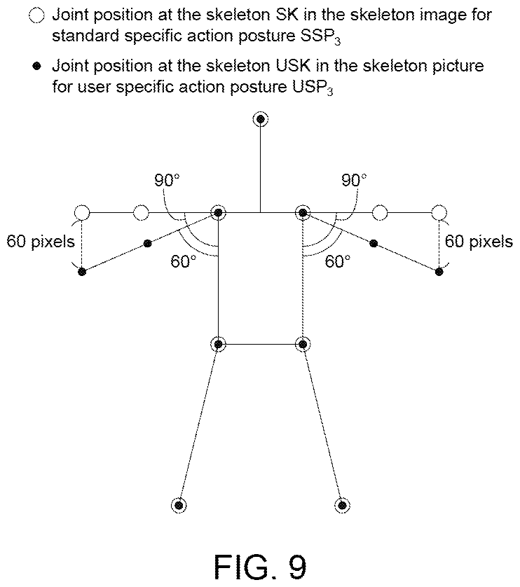

Referring to FIG. 9, a schematic diagram of comparing the skeleton picture for user specific action posture USP₃ with the skeleton image for standard specific action posture SSP₃ to generate a comparison result CR according to an embodiment of the present invention is shown. For the convenience of description, each hollow circle ◯ represents a joint position at the skeleton SK in the skeleton image for standard specific action posture SSP₃, and each solid circle ● represents a joint position at the skeleton USK in the skeleton picture for user specific action posture USP₃. In the present embodiment, the key point of the action in the skeleton image for standard specific action posture SSP₃ is the arm position and the angle between the shoulder joint and the torso. Therefore, the skeleton comparison module 160 compares the arm position at the skeleton USK in the skeleton pictures for user specific action posture USP₃ with the arm position at the skeleton SK in the skeleton image for standard specific action posture SSP₃ and obtains a position comparison result showing that the arm positions at the two skeleton pictures have a distance difference of 60 pixels. The pixels on the picture can be converted to the distance on the picture (such as centimeters). The editor can predetermine a position threshold (such as 50 pixels), and the skeleton comparison module 160 can determine whether the distance difference is tolerable. The skeleton comparison module 160 compares the angle (60°) between the shoulder joint and the torso at the skeleton USK in the skeleton picture for user specific action posture USP₃ with the angle (90°) between the shoulder joint and the torso at the skeleton USK in the skeleton image for standard specific action posture SSP₃ and obtains an angle comparison result showing that the two angles have a difference of 30°. Similarly, the editor can predetermine an angle threshold (such as 10°), and the skeleton comparison module 160 can determine whether the angle difference is tolerable.

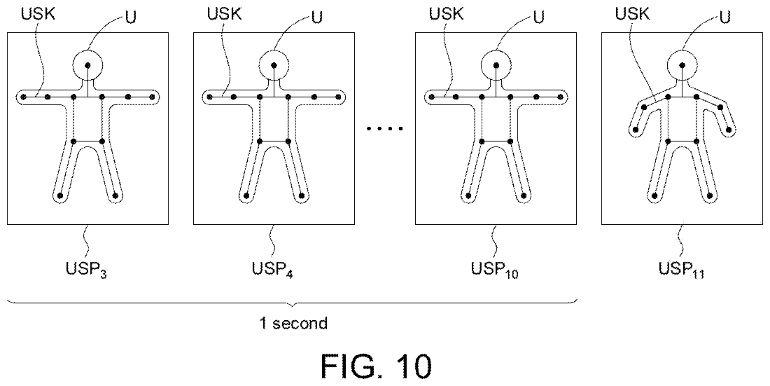

Referring to FIG. 10, a schematic diagram of a user action posture skeleton video according to another embodiment of the present invention is shown. As shown in FIG. 10, the user action posture skeleton video USV is a video with marked skeleton USK. The user action posture skeleton video USV includes several skeleton pictures for user specific action posture USP₃, USP₄ . . . USP₁₀ and USP₁₁. The skeleton pictures for user specific action posture USP₃, USP₄ . . . USP₁₀ and USP₁₁ correspond to the skeleton images for standard specific action posture. Firstly, the skeleton comparison module 160 determines whether the arm position at the skeleton USK in the skeleton picture for user specific action posture USP₃ is the same as the arm position at the skeleton SK in the skeleton image for standard specific action posture SSP₃. If the arm positions at the skeleton USK and the skeleton SK are the same, the skeleton comparison module 160 calculates a time interval in the user action posture skeleton video USV between the starting time of the skeleton picture and the ending time of the skeleton picture in which the arm of the skeleton USK performs the user specific action posture to obtain the duration for which the user performs the user specific action posture. As shown in FIG. 10, the skeleton comparison module 160 obtains the starting skeleton picture USP₃ and the ending skeleton picture USP₁₀ in which the user keeps his/her arms parallel from several skeleton pictures for user specific action posture USP₃, USP₄ . . . USP₁₀ and USP₁₁, and obtains that the user keeps his/her arms parallel for 1 second. The skeleton comparison module 160 further compares the duration for which the user keeps his/her arms parallel with the time guidance information “Keep the arms parallel for 2 seconds” and obtains a time comparison result showing that the two durations have a difference of 1 second.

In step S240, one of the action posture guidance information GI is selected from the guidance knowledge base 131 by the action guidance module 170 according to the comparison result CR. When the action guidance module 170 determines that the position comparison result (60 pixels) exceeds a position threshold (such as 50 pixels), the action guidance module 170 selects the position guidance information “The arms should be parallel to the ground” corresponding to the skeleton image for standard specific action posture SSP₃ from the guidance knowledge base 131. When the action guidance module 170 determines that the angle comparison result (30°) exceeds an angle threshold (such as 5°), the action guidance module 170 selects the angle guidance information “Each shoulder joint needs to form an angle of 90° with the torso” corresponding to the skeleton image for standard specific action posture SSP₃ from the guidance knowledge base 131. When the action guidance module 170 determines that the time comparison result (such as 1 second) exceeds a time threshold (such as 0.5 seconds), the action guidance module 170 selects a time guidance information “Keep the arms parallel for 2 seconds” corresponding to the skeleton image for standard specific action posture SSP₃ from the guidance knowledge base 131. When the action guidance module 170 determines that the speed comparison result (such as 0.5 m/s) exceeds a speed threshold (such as 0.4 m/s), the action guidance module 170 selects the speed guidance information “Control the arm action to a speed of 0.4 m/s” corresponding to the skeleton image for standard specific action posture SSP₃ from the guidance knowledge base 131. Furthermore, corresponding feedback information “Please slow down” or “Please speed up” in response to the speed being too fast or too slow can be created. When the action guidance module 170 determines that the position comparison result does not exceed the position threshold, the angle comparison result does not exceed the angle threshold, the time comparison result does not exceed the time threshold, and the speed comparison result does not exceed the speed threshold, the action guidance module 170 does not select the action posture guidance information GI. It should be noted that the position threshold, the angle threshold, the time threshold and the speed threshold of the present invention are not limited to the above exemplifications and can be adjusted according to the action posture.

In an embodiment, the guidance system for action postures 100 further includes an exercise repetition calculation module 190. The exercise repetition calculation module 190 can be a processing circuit or a processing chip. When the position comparison result does not exceed the position threshold and the angle comparison result does not exceed the angle threshold, the exercise repetition calculation module 190 records an exercise repetition.

In step S250, the selected action posture guidance information GI is shown on the display 180. In step S240, if the action posture guidance information GI selected from the guidance knowledge base 131 by the action guidance module 170 is the position guidance information “The arms should be parallel to the ground”, the display 180 shows “the arms should be parallel to the ground”. If the action posture guidance information GI selected from the guidance knowledge base 131 by the action guidance module 170 is the angle guidance information “Each shoulder joint needs to form an angle of 90° with the torso”, the display 180 shows “each shoulder joint needs to form an angle of 90° with the torso”. If the action posture guidance information GI selected from the guidance knowledge base 131 by the action guidance module 170 is the speed guidance information “The arm action needs to be lasted for 2 seconds”, the display 180 shows “The arm action needs to be lasted for 2 seconds”. If the action guidance module 170 does not select the action posture guidance information GI, the display 180 shows a prompt information “Perfect action” to indicate that the user's action posture is correct.

Thus, the guidance system and method for action postures of the present invention are capable of determining whether the user's action posture is correct according to the position, the angle and the speed of the action posture when the user is doing exercise or training, and providing the user with action posture guidance information which helps the user to avoid having incorrect action posture during the exercise or training process or ending up with an undesired effect or sports injury.

While the invention has been described by way of example and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A guidance system for action postures, comprising:
a first skeleton recognition model for generating a standard skeleton video according to a standard action posture video, wherein the standard action posture video comprises a standard specific action posture of a person, and the standard skeleton video comprises a plurality of skeleton images for standard specific action posture corresponding to the standard specific action posture;
an editing device for receiving a command of an editor to generate at least one action posture guidance information corresponding to the skeleton images for standard specific action posture; and
a storage device for storing the skeleton images for standard specific action posture and the at least one action posture guidance information corresponding to the skeleton images for standard specific action posture.

2. The guidance system according to claim 1, further comprising:
a communication module for receiving the standard action posture video.

3. The guidance system according to claim 1, wherein the storage device further comprises a guidance knowledge base for storing the skeleton images for standard specific action posture and the at least one action posture guidance information corresponding to the skeleton images for standard specific action posture.

4. The guidance system according to claim 3, further comprising:
a capturing device for capturing a user action posture of a user to generate a user live streaming;
a second skeleton recognition model for generating a user action posture skeleton video according to the user live streaming, wherein the user live streaming comprises a user specific action posture of the user, and the user action posture skeleton video comprises a plurality of skeleton pictures for user specific action posture corresponding to the user specific action posture;
a skeleton comparison module for comparing the skeleton pictures for user specific action posture with the skeleton images for standard specific action posture to generate a comparison result;
an action guidance module for selecting one of the action posture guidance information from the guidance knowledge base according to the comparison result; and
a display for showing the selected action posture guidance information.

5. The guidance system according to claim 4, wherein the selected action posture guidance information comprises at least one of a position guidance information, an angle guidance information, a time guidance information and a speed guidance information.

6. The guidance system according to claim 4, wherein the comparison result comprises at least one of a position comparison result, an angle comparison result, a time comparison result and a speed comparison result.

7. The guidance system according to claim 6, wherein when the position comparison result does not exceed a position threshold, the angle comparison result does not exceed an angle threshold, the time comparison result does not exceed a time threshold and the speed comparison result does not exceed a speed threshold, the action guidance module does not select any of the action posture guidance information, and the display shows a prompt information to indicate that the user's action posture is correct.

8. The guidance system according to claim 6, further comprising:
an exercise repetition calculation module for recording an exercise repetition when the position comparison result does not exceed a position threshold and the angle comparison result does not exceed an angle threshold.

9. The guidance system according to claim 1, wherein the editing device comprises an operation interface on which a standard skeleton video is displayed, providing the editor to select one of the skeleton images for standard specific action posture from the standard skeleton video and further to perform information marking on the selected skeleton image for standard specific action posture, wherein a skeleton in the skeleton image for standard specific action posture comprises a plurality of limbs and a plurality of joints, and the information mark comprises a position mark or a time mark on any of the limbs, or an angle mark on any of the joints and the limb connected thereto.

10. The guidance system according to claim 9, wherein the operation interface further provides the editor to edit the action posture guidance information according to the information mark.

11. A guidance method for action postures, comprising:
generating a standard skeleton video according to a standard action posture video, wherein the standard action posture video comprises a standard specific action posture of a person, and the standard skeleton video comprises a plurality of skeleton images for standard specific action posture corresponding to the standard specific action posture;
receiving a command of an editor to generate at least one action posture guidance information corresponding to the skeleton images for standard specific action posture; and
storing the skeleton images for standard specific action posture and the at least one action posture guidance information corresponding to the skeleton images for standard specific action posture.

12. The guidance method according to claim 11, further comprising:
receiving the standard action posture video through a communication module.

13. The guidance method according to claim 11, further comprising:
storing the skeleton images for standard specific action posture and the at least one action posture guidance information corresponding to the skeleton images for standard specific action posture to a guidance knowledge base.

14. The guidance method according to claim 13, further comprising:
capturing a user action posture of a user to generate a user live streaming;
generating a user action posture skeleton video according to the user live streaming, wherein the user live streaming comprises a user specific action posture of the user, and the user action posture skeleton video comprises a plurality of skeleton pictures for user specific action posture corresponding to the user specific action posture;
comparing the skeleton pictures for user specific action posture with the skeleton images for standard specific action posture to generate a comparison result;
selecting one of the action posture guidance information from the guidance knowledge base according to the comparison result; and
showing the selected action posture guidance information.

15. The guidance method according to claim 14, wherein the selected action posture guidance information comprises at least one of a position guidance information, an angle guidance information, a time guidance information and a speed guidance information.

16. The guidance method according to claim 14, wherein the comparison result comprises at least one of a position comparison result, an angle comparison result, a time comparison result and a speed comparison result.

17. The guidance method according to claim 16, wherein when the position comparison result does not exceed a position threshold, the angle comparison result does not exceed an angle threshold, the time comparison result does not exceed a time threshold and the speed comparison result does not exceed a speed threshold, none of the action posture guidance information is selected, and a prompt information is shown to indicate that the user's action posture is correct.

18. The guidance method according to claim 16, further comprising:
recording an exercise repetition when the position comparison result does not exceed a position threshold and the angle comparison result does not exceed an angle threshold.

19. The guidance method according to claim 11, further comprising:
playing the standard skeleton video on an operation interface, which provides the editor to select one of the skeleton images standard specific action posture from the standard skeleton video and to perform information marking on the selected skeleton image standard specific action posture;
wherein a skeleton in the selected skeleton image standard specific action posture comprises a plurality of limbs and a plurality of joints, and the information mark comprises a position mark or a time mark on any of the limbs, or an angle mark on any of the joints and the limb connected thereto.

20. The guidance method according to claim 19, wherein the operation interface further provides the editor to edit the action posture guidance information according to the information mark.
